(19) United States
(12) Patent Application Publication
(10) Pub. No.: US 2009/0014015 A1
(43) Pub. Date: Jan. 15, 2009

(54) INTRAOPERATIVE DOSIMETRY FOR PROSTATE BRACHYTHERAPY USING TRANSRECTAL ULTRASOUND AND X-RAY FLUOROSCOPY

(75) Inventors: Ismail Tutar, Seattle, WA (US); Paul S. Cho, Shoreline, WA (US); Sreeram Narayanan, Pleasant Hill, CA (US); Sayan Pathak, Kirkland, WA (US); Lixin Gong, Seattle, WA (US); Junqing Shang, Seattle, WA (US); Yongmin Kim, Seattle, WA (US)

Correspondence Address: LAW OFFICES OF RONALD M ANDERSON, 600 108TH AVE, NE, SUITE 507, BELLEVUE, WA, 98004, US

(73) Assignee: University of Washington, Seattle, WA (US)

(21) Appl. No.: 12/103,864

(22) Filed: Apr. 16, 2008

Related U.S. Application Data
(60) Provisional application No. 60/912,371, filed on Apr. 17, 2007.

(51) Int. Cl. A61M 36/12

(52) U.S. Cl. 128/898; 600/7

(57) ABSTRACT

While performing prostate brachytherapy in which radioactive seeds are implanted into the prostate, both X-ray and transrectal ultrasound (TRUS) data are collected. The X-ray data indicate all of the implanted seeds, but not their relative position within the prostate. The TRUS data correspond to both axial and longitudinal ultrasound images. A subset of the implanted seeds can be automatically (or manually) detected in the TRUS data (using longitudinal ultrasound images), and the prostate is indicated in the TRUS data (using axial ultrasound images). Registration is performed between the X-ray and TRUS data, thereby identifying the disposition of all implanted seeds within the prostate, enabling dosimetry to be intraoperatively determined. A medical practitioner can thus determine whether and where to implant additional seeds to achieve a desired optimal dosage and do so before the prostate brachytherapy procedure is concluded.

### RELATED APPLICATIONS

This application is based on a prior copending provisional application Ser. No. 60/912,371, filed on Apr. 17, 2007, the benefit of the filing date of which is hereby claimed under 35 U.S.C. § 119(e).

### GOVERNMENT RIGHTS

This invention was made with government support under Contract No. DAMD 17-03-1-0033 awarded by the Department of Defense (DOD). The government has certain rights in the invention.

### BACKGROUND

Prostate cancer is a commonly diagnosed cancer in men in the United States, second only to skin cancer. One of the most commonly used treatment modalities for prostate cancer is transperineal interstitial permanent prostate brachytherapy (TIPPB), which involves permanent implantation of 35-140 radioactive seeds into the prostate gland according to a preplan. Prostate brachytherapy is generally considered to be an effective treatment modality for early stage prostate cancer. However, as with many medical therapies, the success rates have varied markedly between medical practitioners. At least part of the difference in clinical outcomes is related to technical differences or implant quality. There is a complex, largely undefined relationship between dosimetric parameters and cancer control rates. The most widely reported parameters related to biochemical cancer control with radioactive seeds are the D90 dose (the minimum dose delivered to 90% of the prostate), and the V100 dose (the percent of the postimplant prostate volume covered by the prescription dose). These quality parameters are traditionally derived from a postimplant computed tomography (CT) scan.

During a prostate brachytherapy procedure, transrectal ultrasound (TRUS) and fluoroscopy imaging modalities complement each other by separately providing good visualization of the soft tissue at the treatment site, and of the implanted seeds, respectively, enabling the medical practitioner to separately view images of the prostate gland and images of the implanted seeds. Due to several factors such as prostate swelling, needle deviation from the planned direction, seed movement along the needle tracks, and change in patient positioning, some deviations from the preplan used for implant of the seeds are inevitable. Therefore, the postimplant CT scan is performed to compute the dose that will be received by the prostate with the initial implanted seeds. If inadequate isodose patterns are detected in the CT scan, a patient is considered for further treatment, such as an external beam radiation therapy or a second implant. However, a return trip to the operating room for implant of additional seeds clearly inconveniences the patient and increases the cost of healthcare. Instead, it would be desirable to intraoperatively assess the seed implant dosage parameters or isodose patterns at the time of the initial seed implant, and implant additional seeds as appropriate at that time before the initial prostate brachytherapy is completed. However, conventional prostate brachytherapy techniques do not provide any acceptable approach for evaluating the dosage parameters during the actual implant procedure.

If dosimetry evaluation could be performed intraoperatively, physicians could implant additional seeds into the under-dosed portions of the prostate while the patient remains on the operating table, thus obviating further treatment at a later time. Several researchers have attempted to perform dosimetry using ultrasound imaging modality only. However, ultrasound imaging is suboptimal in terms of seed visibility, and at least 95% of the implanted seeds need to be detected to provide an accurate estimation of dose parameters. Although some improvement in seed visibility has been achieved by using an alternating magnetic field, vibroacoustography, transurethral ultrasound, 3-D TRUS imaging, suture-mounted seeds, and seeds with a textured surface, all of these methods have either fallen significantly short of the required seed detection rate or have not been evaluated with real clinical data to determine their efficacy.

Seed detection in ultrasound images is clearly very important as one of the steps necessary to enable a clinician to evaluate the dosage that will be delivered by implanted seeds. No image modality is suitable for both implanted seeds and the prostate gland in the operating room. Both the fluoroscopy and TRUS images can be produced at that time, but currently, the two types of images are not easily correlated. In the fluoroscope images, soft tissues are not delineated, but the seeds are very clear; while in TRUS images, the prostate boundaries are clearly evident, but only part of the seed images appears and can be confused with non-seed structures, such as gas bubbles and calcifications, or other image elements or artifacts.

Accordingly, seed detection in TRUS images is an active area of medical image analysis. Research in this area has resulted in the development of an algorithm for seed detection based on the constant false alarm rate detection, and also, an algorithm for three-dimensional (3-D) seed detection. Another algorithm for seed detection has been developed that uses ultrasound radio frequency signals. However, as reported in the prior art, these experiments were not based on analysis of real clinical images, but instead, on phantom images. Still another prior art approach that did evaluate clinical data made use of the seed implant needle as an external fiducial, but that approach is believed likely too cumbersome for clinical use.

### Detecting Implanted Seeds in TRUS Images

Based on empirical experience, only about 20% of the implanted seeds can typically be detected in TRUS images acquired by using the longitudinal transducer of a bi-planar TRUS probe. Due to the poor seed visibility in TRUS images, some researchers have used the locations of needle tips when seeds were placed to compute an estimate of dosimetry. However, this method is inaccurate, since it does not account for prostate swelling or seed movement.

In contrast to TRUS images, all of the implanted seeds are evident in three or more fluoroscopy images acquired from different angles. Since TRUS complements fluoroscopy by providing good visualization of the soft tissue, the combination of these two imaging modalities could enable effective and efficient determination of intraoperative dosimetry. However, a suitable TRUS-fluoroscopy registration method has not been developed or suggested in the prior art that can determine all of the seed locations in relation to the prostate boundaries.

To perform TRUS-fluoroscopy registration, markers that are visible in both imaging modalities, such as needle tips and radio-opaque fiducial markers, have been used. However, these methods are cumbersome and further complicate the brachytherapy procedure, since there is barely enough space inside a seed-implanted prostate to insert needles or implant fiducial markers. Furthermore, these markers are not used in routine seed implant procedures and increase the cost.

Other researchers have proposed the use of embedded markers on a modified ultrasound probe and fiducial markers above the abdomen. These external fiducial markers can be identified in fluoroscopy images and are assumed to be in known calibrated pose with respect to the TRUS coordinate system. But, the distance between the prostate and fiducial markers leads to error propagation, thus reducing the accuracy in registering seeds to the prostate boundaries. If this distance is too great, fiducial markers may not even be visible in fluoroscopy images acquired from oblique views. External fiducial marker-based approaches are also very sensitive to patient motion, which is a major limitation, especially for procedures performed under local anesthesia, since patients often move during image acquisition.

Therefore, it would clearly be desirable to develop an efficient approach for identifying seeds in TRUS images, and for then performing a registration between the seeds in the fluoroscopic and TRUS images. In this manner, it should be possible to find the relative locations of seeds within the prostate boundary to evaluate dosage parameters. Furthermore, it would be preferable to automatically detect the seeds in fluoroscopic images, either manually or automatically detect the locations of at least some of the seeds in the TRUS images, and then automatically perform the registration between the two types of images using the seed location data from each. A medical practitioner should then be able to intraoperatively determine if more seeds need to be implanted and implant the additional seeds where needed.

### SUMMARY

In consideration of the preceding discussion, a method has been developed for performing a prostate brachytherapy procedure on a patient, in which radioactive seeds are implanted at a treatment site in the patient, and enabling intraoperative determination of a dosage parameter for implanted seeds during the brachytherapy procedure. After implanting a plurality of radioactive seeds at the treatment site, X-ray data are acquired that identify locations of a majority (typically all) of the implanted seeds. In addition, ultrasound data are collected that identify a location of the prostate, as well as the locations of each of only a minority of the implanted seeds relative to the prostate. It should be noted, that the minority of implanted seeds and their locations relative to the prostate are identified without solely relying on cross-sectional images of the seeds. Indeed, the seeds are more evident in longitudinal ultrasound images of the treatment site. Next, the method registers the X-ray data with the ultrasound data to determine the locations of the majority of the implanted seeds relative to the prostate. The registration of the seeds in the two different types of images enables a dosage parameter for the implanted seeds to be determined, based on the locations of the majority of the seeds relative to the prostate.

The longitudinal ultrasound images are along an axis that is generally aligned with a longitudinal axis of each implanted seed. The step of identifying the minority of seeds can be done automatically, as follows. Bright structures in the longitudinal ultrasound images are readily identified because they have a reflected ultrasound power level that is substantially greater than a background ultrasound power level. The structures thus appear as bright objects in the longitudinal images. The method then identifies any of the bright objects identified in the longitudinal ultrasound images that correspond to line structures and detects each of the line structures that includes an adjacent mirror structure reflection. The presence of an adjacent mirror structure reflection is an indication that the line structure is an implanted seed.

The step of acquiring the ultrasound data can include the step of rectally inserting an ultrasound probe to enable transrectal ultrasound (TRUS) image data of the treatment site to be collected in orthogonally different orientations. Alternatively, a 3-D rectally inserted probe can be employed to produce volumetric ultrasound data that correspond to both axial and longitudinal images of the prostate. As a further alternative, an ultrasound probe can be inserted through a patient's urethra to enable transurethral ultrasound images to be collected in orthogonally different orientations.

The step of acquiring X-ray data includes the step of collecting a plurality of fluoroscopic images of the treatment site at different angular orientations, after the seeds have been implanted, so that spatial locations of the seeds can be determined. This step can be implemented automatically or manually.

The step of determining a dosage parameter for the implanted seeds can include the step of determining isodose contours for the implanted seeds relative to the prostate. In an exemplary embodiment, the method further includes the step of displaying the isodose contours of the implanted seeds in relation to the prostate. A medical practitioner viewing the isodose contours can then intraoperatively determine whether additional seeds should be implanted and if so, where the additional seeds should be implanted during the procedure to achieve a desired radiation dosage effect on the prostate.

The step of registering the X-ray data with the ultrasound data can include the step of iteratively performing an optimization loop that transforms the X-ray data and correlates the resulting transformed X-ray data with the ultrasound data. The step of iteratively performing the optimization loop can include the steps of performing a rigid body transformation of the X-ray data based on a current pose parameter set, thereby producing the transformed X-ray data, and computing an optimal assignment of the transformed X-ray data to the ultrasound data. Until a solution to the optimal assignment converges, the method repetitively adjusts the current pose parameter set and carries out the steps of performing the rigid body transformation, and computing the optimal assignment.

The step of registering the X-ray data with the ultrasound data further can include the steps of recording the solution that has converged and the associated costs of the solution. Until the optimization loop has been entered a predefined maximum number of times that is greater than a predefined maximum number of iterations, a new pose parameter set is sampled for use as the current pose parameter set. The optimization loop is then reentered with the new pose parameter set, which now comprises the current pose parameter set. The steps comprising the optimization loop are then repeated until the solution again converges. A current solution that has converged is recorded, along with a current associated cost of said solution. The iterative process is finally concluded, returning the current solution and the current associated cost, after the optimization loop has been entered more times than the predefined maximum number of iterations.

Another aspect of this novel approach is directed to an exemplary system for performing a prostate brachytherapy procedure on a patient. The system includes an ultrasound system having a probe configured to produce ultrasound data corresponding to volumetric images or both axial and longitudinal ultrasound images of the treatment site. The longitudinal images are generally aligned with a longitudinal axis of seeds that have been implanted, and the axial images are generally transverse to the longitudinal axis of the seeds. The ultrasound data can be evaluated to indicate a location of only a minority of the seeds that have been implanted relative to the prostate of the patient.

Also included in the system is a fluoroscope that is configured for producing X-ray data for the treatment site, indicating a majority of the seeds that have been implanted. A computing device is provided for processing the ultrasound data and the X-ray data and carrying out a plurality of functions. These functions are generally consistent with the steps of the method described above.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1A is a schematic view of the apparatus used in prostate brachytherapy for implanting radioactive seeds and imaging the prostate and at least some of the seeds that have been implanted using TRUS, and for imaging all of the implanted seeds using a fluoroscope;

FIG. 1B is an isometric view of a template assembly used for guiding needles while implanting radioactive seeds during the prostate brachytherapy procedure;

FIG. 2A is a schematic illustration showing the orientation of axial TRUS images, which are generally transverse to the longitudinal axis of the elongate implanted seeds;

FIG. 2B is a schematic illustration showing the orientation of rotational TRUS images, which are generally aligned with and rotated around the longitudinal axis of a TRUS probe at different angular positions;

FIG. 2C is a schematic illustration showing the orientation of coronal TRUS images, which are an alternative to the rotational images of FIG. 2B;

FIG. 2D is a schematic illustration showing the orientation of sagittal images, which are yet another alternative to the rotational images of FIG. 2B;

FIG. 3 is an exemplary rotational TRUS image showing implanted seeds, as well as illustrating dark seeds, speckles, calcification, and air bubbles, which make it difficult to determine the location of implanted seeds in the prostate within this type of image;

FIG. 4 is a flowchart illustrating exemplary logical steps employed in intraoperatively determining the dosimetry of implanted radioactive seeds in accord with the present novel approach;

FIG. 5 is a flowchart illustrating exemplary logical steps employed for automatically identifying the location of at least a minority of the implanted seeds in the rotational (or other type of longitudinal) TRUS images;

FIG. 6 is a flowchart illustrating exemplary logical steps of the iterative optimization algorithm that is implemented to determine the correspondence between the fluoroscopic and TRUS images, to enable determining dosage parameters;

FIG. 7 illustrates an exemplary longitudinal TRUS image and reflected ultrasound power peaks detected in the image to show how vertical lines within a longitudinal TRUS image are treated as a scan line of an ultrasound beam, and how, using a 6-pixel wide window, local peaks are used to detect bright line structures;

FIG. 8 illustrates four exemplary 16-pixel wide windows in which the specular reflection from the smooth seed surface produces adjacent bright structures indicative of an implanted seed;

FIG. 9 illustrates four exemplary 16-pixel wide windows in which the bright structures are probable calcifications and/or air bubbles (e.g., air bubbles trapped along the needle track), which exhibit a rough surface that does not produce a specular reflection;

FIG. 10 is a graphic view illustrating an exemplary model of the mirror structure produced by synthesizing an image pattern;

FIG. 11 illustrates schematically an example showing how a two-dimensional (2-D) image is extended to a one-dimensional signal;

FIG. 12 illustrates how local contrast in an image is used to distinguish between tissue and an implanted seed, by comparison of the average brightness with that of two 2-D windows above and below a prospective seed structure;

FIGS. 13A, 13B, and 13C, respectively illustrate examples of a longitudinal TRUS image after respectively carrying out the first, second, and third steps of the algorithm employed to automatically identify a seed structure, wherein the white circles show the bright line structures in FIG. 13B (some of which are calcification structures and/or air bubbles), and the seed structures in FIG. 13C;

FIG. 14 illustrates exemplary corresponding TRUS (left images) and CT (right images) cross-sectional images showing isodose contours for seeds implanted in a prostate and showing (bottom images) 3-D lateral views of the isodose cloud;

FIG. 15 illustrates exemplary corresponding TRUS cross-sections before additional seeds are added (left column), and after the dose has been touched-up by adding additional seeds where appropriate (center column) compared to CT cross-sections, and showing (bottom images) 3-D lateral views of the isodose cloud;

FIG. 16 is a functional block diagram of an exemplary system for imaging a treatment site during prostate brachytherapy in which intraoperative determination of dosage is carried out; and

FIG. 17 is a functional block diagram of an exemplary generally conventional computing device, such as a personal computer (PC) that can be employed as a controller in the system of FIG. 16 to control the imaging system and to enable intraoperative dosage determination.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

### Apparatus for Administering Prostate Brachytherapy

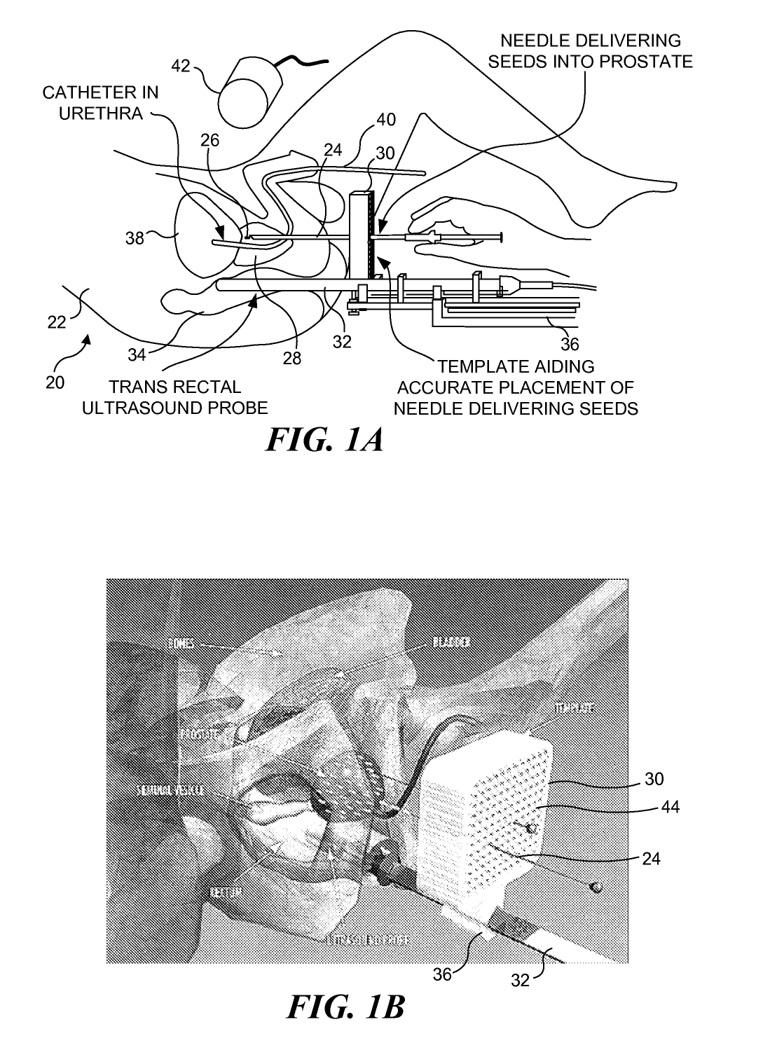

FIG. 1A is schematic diagram 20 illustrating a patient 22 undergoing prostate brachytherapy. In connection with this Figure, the present approach facilitates intraoperative dosimetry, i.e., enables evaluation of radiation dosage and seed placement while the patient remains positioned for the radioactive seed implant procedure, so that additional seeds can readily be implanted, as necessary to achieve optimal radiation dosage and seed distribution in the patient's prostate. A conventional seed emplacement needle 24 is employed for implanting a plurality of generally elongate radioactive seeds 26 along a track of the needle as the needle is withdrawn. The seeds, which are cylindrical and about 4.8 mm long, with a radius of about 0.4 mm, are forced from the needle at intervals along the track within prostate 28. An array for assisting the medical practitioner is defined by a template 30, which includes orifices 44 through which needle 24 is inserted to aid in the accurate positioning of the needle for delivering the seeds, as illustrated in FIG. 1B. The seeds are thus delivered along generally parallel tracks in the prostate and generally along the craniocaudal axis. The medical practitioner is able to view a TRUS image of prostate 28 produced by an ultrasound probe 32 that is inserted into rectum 34 of the patient. The ultrasound probe is positioned by a stepper drive 36 (which also includes an encoder for producing a signal indicative of the axial and rotational position of the ultrasound probe). Also shown in the Figure is a catheter 40, which extends through the urethra into bladder 38 so that urine can be drained. A fluoroscope 42 is provided to produce fluoroscopic images of the prostate at a plurality of different angular positions (e.g., −15°, 0°, and +15°), in which all of the implanted seeds are clearly visible. However, the fluoroscopic images do not show the prostate outlines, since it is soft tissue. Accordingly, it will be evident that the present approach, which enables the registration of the ultrasound images and the fluoroscopic images, enables the medical practitioner to more accurately determine the positioning of the seeds within the prostate, and thus, the dosage parameters of the seeds implanted. Based on these dosage parameters, the medical practitioner can determine where to implant additional seeds in the prostate to achieve an optimal desired result.

In one embodiment, a Hitachi Corporation HI VISION 5500™ ultrasound machine (not shown in this Figure) and a U-533™ biplanar TRUS probe were used with a Civco EXII™ stepper drive. The fluoroscopic images were produced by an Elektra SLS-14™ fluoroscope. The biplanar TRUS probe that was used includes both a longitudinal transducer and an axial transducer, so that orthogonal views of the prostate can be produced. The data acquisition procedure positions the biplanar TRUS probe within the patient using the stepper driver and produces data corresponding to the axial and rotational angular position of the TRUS probe provided from a position encoder on the stepper drive, thus providing geometrical information for all acquired ultrasound images, with respect to a reference coordinate system.

Using the longitudinal transducer of the TRUS probe, a first set of images are acquired by rotating the longitudinal ultrasound transducer in either a clockwise or counterclockwise direction about its longitudinal axis, while the corresponding angle and depth information provided by the position encoder are recorded. This first set of images are referred to herein, as “rotational images.” Rotational images can be acquired at 1° angular increments (or at other angular increments, as desired). By using the axial transducer of the TRUS probe, a second set of images can be captured at 1 mm increments along the longitudinal axis, using the stepper driver to move the ultrasound axial transducer in the craniocaudal direction. The corresponding depth and angle information provided by the position encoder of the stepper drive is recorded for these two sets of images. The images in the second set of images are referred to herein as “axial images.”

In conventional prostate brachytherapy, only the axial ultrasound images are typically used, and generally, no attempt is made to identify seeds in these ultrasound images. In axial images, the seeds would be imaged in transverse cross-section and thus, are barely visible as dots—unless fiducial markers are used. In contrast, the rotational (or other longitudinal) images produced by the longitudinal transducer in the biplanar TRUS probe enable a subset (e.g., 20%) of the total number of seeds implanted to be detected. While the seeds are more visible in their longitudinal aspect than in their cross-sectional aspect, noise, other artifacts, and poor visibility of the seeds in the ultrasound rotational or other longitudinal images make detection difficult. The detection of the minority of the total seeds comprising this subset can be done either manually or automatically, as explained below.

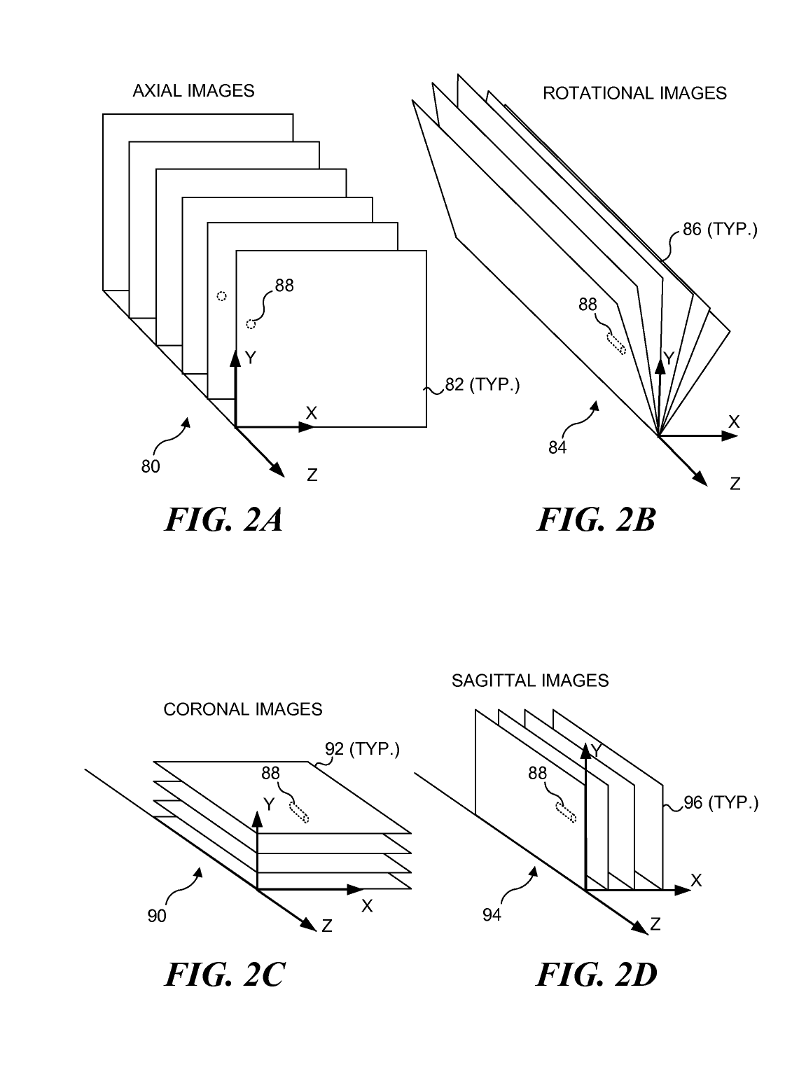

FIGS. 2A and 2B respectively illustrate a set 80 of axial images 82, and a set 84 of rotational images 86. As an alternative to using the rotational images of FIG. 2B, either a set 90 of coronal images 92 or a set 94 of sagittal images 96 may be used, as respectively illustrated in FIGS. 2C and 2D. Indeed, any set of ultrasound images extending along the Z direction can be used to improve seed detection rate compared to the axial images of FIG. 2A. Furthermore, such images can also be obtained by using 3-D TRUS (mechanically or electronically steered) to produce 3-D volumetric scans, or other well-known 2-D/3-D ultrasound imaging techniques. It must also be emphasized that other similar ultrasound imaging techniques can be employed, such as transurethral ultrasound imaging of the treatment site instead of TRUS imaging. The present approach is clearly applicable to transurethral ultrasound imaging, in much the same manner as to TRUS imaging, in combination with X-ray imaging of the treatment site.

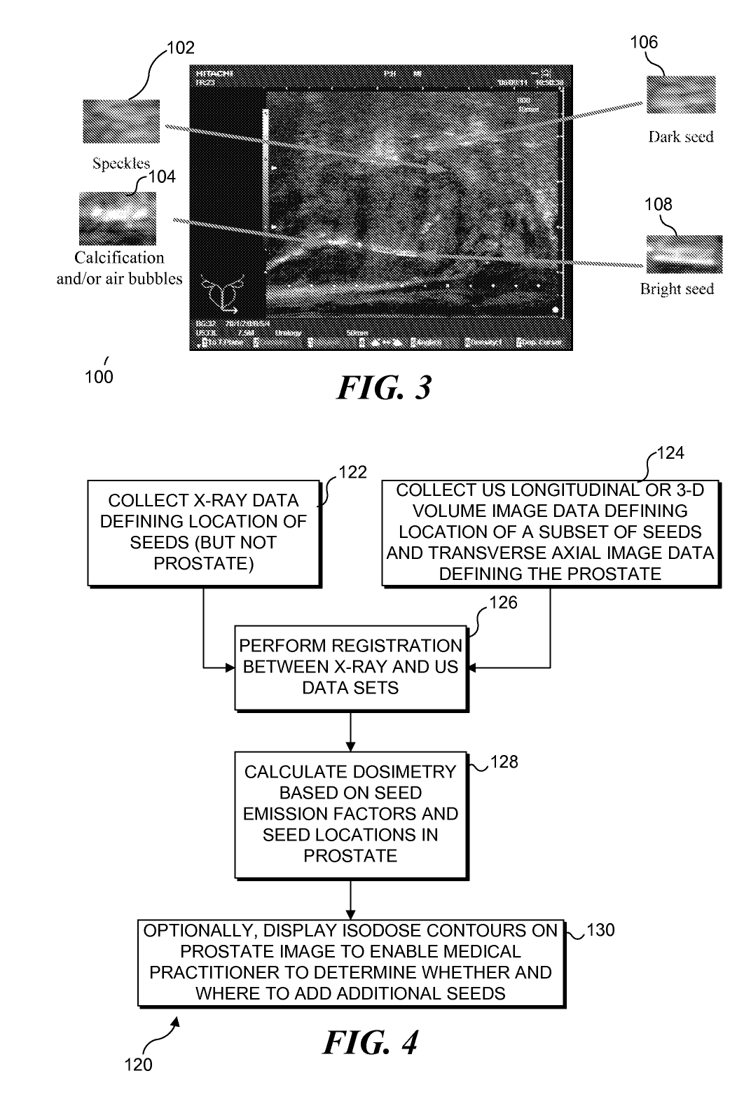

It will be evident that the cross-sectional aspect of a seed 88 in the axial images is much less evident than the longitudinal aspect of the seed in the rotational or other types of longitudinal images. However, as will be evident in an exemplary rotational TRUS image 100 of the prostate shown in FIG. 3, even though the seeds are visually more evident than in the axial TRUS images, it can still be difficult to identify seed locations in the rotational images. In the rotational TRUS images, each radioactive seed appears as a bright line structure, which has a length and width that depends upon the spatial resolution of the displayed image. In a sagittal TRUS image with a resolution of about 0.13 mm/pixel, each bright line structures is typically about 37 pixels long and 6 pixels wide, and has a mirror structure above it, which is caused by the specular reflection of ultrasound at the seed surface. However, visual seed detection is difficult due to the poor image quality in ultrasound images comprising real clinical data. For example, as shown in rotational TRUS image 100, a non-seed structure, which is likely to be a calcification and/or an air bubble 104 in the patient's prostate may also have a bright line structure. Some seeds 106 are relatively dark compared to other seeds 108; air bubbles (not indicated) may be caused by needles; calcifications may be present in some patients, and speckles 102 always exist in an ultrasound image. These artifacts and visual deficiencies in delineating seeds thus make the task of identifying seed locations in the rotational images relatively difficult, whether done manually or automatically.

### Steps for Registering TRUS and Fluoroscopic Images and Calculating Dose Parameters

FIG. 4 illustrates exemplary logical steps 120 that are implemented to enable the registration or correlation of the two different types of images, TRUS and fluoroscopic. The registered images fully define the disposition of the implanted seeds within the prostate, to enable dose related parameters to be determined intraoperatively.

A step 122 provides for collecting X-ray data, for example using a fluoroscope, which indicates the 3-D locations of seeds in space—but not in relation to the prostate. Since the fluoroscopic images do not show the prostate or even its outline, the location of the seeds within these fluoroscopic images alone cannot be related to their position within the prostate, and the dosage parameters for the prostate cannot be determined solely from the fluoroscopic images. Multiple fluoroscopic images are acquired (e.g., −15°, 0° and +15° degrees in case of three) by rotating the gantry of the X-ray fluoroscopy machine about an isocenter (which is in general not a requirement). It may be desirable to collect more or fewer fluoroscopic images at other rotational positions about this axis. An exemplary, well known, seed reconstruction algorithm, which can be used to determine the locations of the implanted seeds, assumes an isocentric imaging geometry, but other techniques can be employed that do not impose this constraint.

In contrast to step 122, a step 124 provides for collecting ultrasound longitudinal (e.g., rotational, coronal, or sagittal images) for the region of the prostate. These TRUS images include sufficient information to enable the locations of a subset (i.e., a minority) of the total number of seeds implanted to be determined. However, the seeds are not all visible in the longitudinal TRUS images, although, it is typically possible to identify the locations of at least 20% of the total number of implanted seeds. Details of an exemplary procedure for identifying the locations of the seeds comprising this subset are discussed below. The axial TRUS image data show seeds only as dots, but provide a clear indication of the limits of the prostate that is geometrically related to the subset of seeds determined from the rotational or other type of longitudinal ultrasound data.

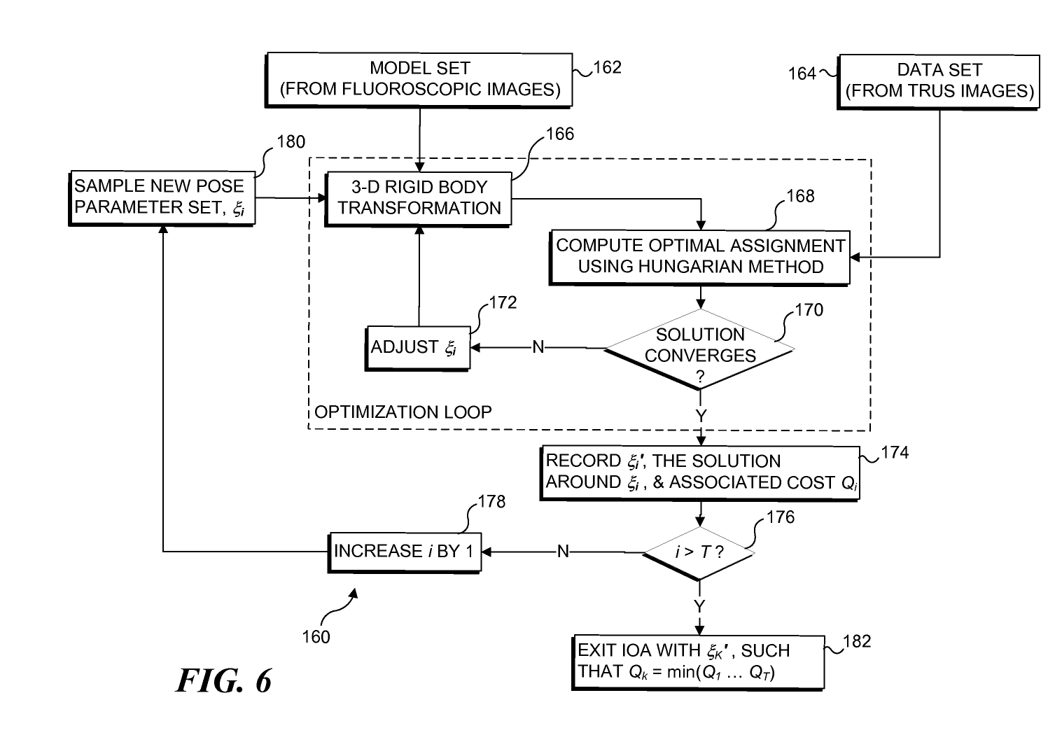

A step 126 then performs a registration between the fluoroscopic or x-ray set of seed locations, referred to as the “N-point model set,” and the TRUS set of seed locations, referred to as the “M-point data set.” An exemplary registration algorithm that is used for this step is referred to as an iterative optimal assignment (IOA) algorithm. The IOA first establishes a one-to-one correspondence between the model set and data set and then computes the sum of error distances between the matching seeds. Further details of the IOA algorithm are discussed below, in connection with FIG. 6.

Once the registration between the two sets of ultrasound and X-ray data has been achieved in step 126, a step 128 calculates the dosimetry based on seed emission factors and seed locations in the prostate defined as a result of registration of the two types of data. Each type of seed that is used for prostate brachytherapy has a characteristic radiation emission that can be used in evaluating the dosage on the prostate, relative to the location of the seeds in the prostate, their density, and other spatial characteristics. Accordingly, this step provides data that indicates the effectiveness of the seeds that have been implanted in achieving the desired radiation therapy of the prostate. Optionally (but likely to be employed in each instance), a step 130 provides for displaying isodose contours on the prostate image to enable a medical practitioner to more readily determine whether additional seeds should be implanted and if so, where the additional seeds should be placed to achieve optimal therapy of the prostate.

### Detection of Implanted Seeds in TRUS Images

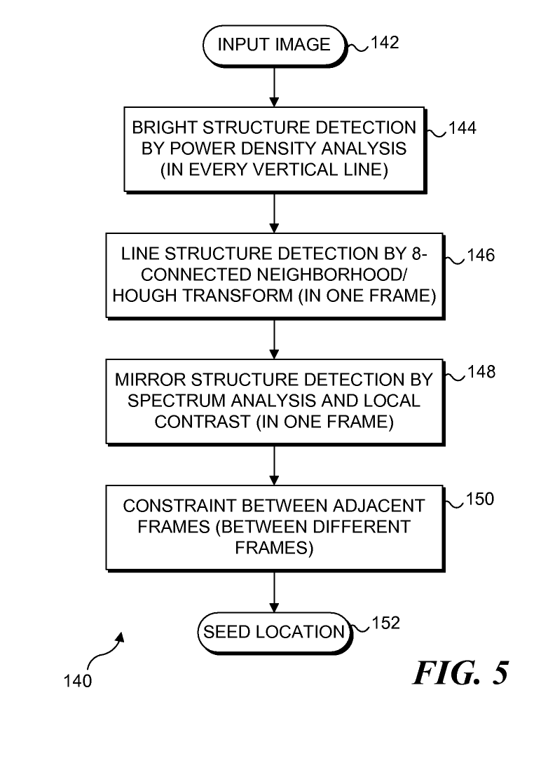

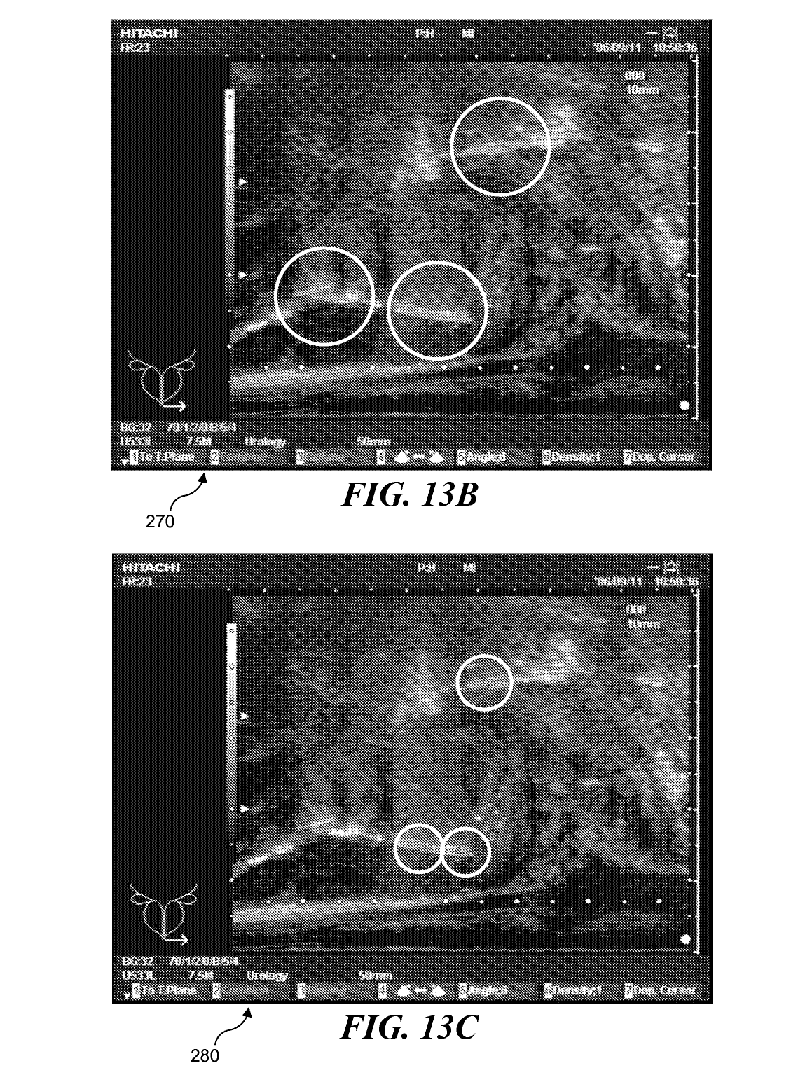

FIG. 5 includes exemplary logical steps 140 for detecting the locations of a subset of the total number of implanted seeds in TRUS images of the prostate. This flow chart is repeated for each TRUS longitudinal image. A step 142 provides for input of a TRUS longitudinal image (e.g., rotational, coronal, or sagittal). A step 144 then detects bright structures in the image by power density analysis of every vertical line in the image. FIG. 13A illustrates an exemplary TRUS image 260 showing the bright structures or objects that have been detected (these bright objects are difficult to see in a gray-scale image). In a step 146, line structures are detected by using an 8-connected neighborhood transform or by using a Hough transform on the result of step 144. FIG. 13B illustrates an example of a TRUS image 270, showing bright structures (indicated within the white circles) that remain after applying step 146 to a TRUS image, to eliminate a substantial number of the bright objects previously identified in FIG. 13A as unlikely to be seeds. Since the seeds are typically of a defined length and are typically implanted by a medical practitioner along a needle track using a template, elements in an image that are too short or are skewed at an angle so that they do not appear along a track can readily be eliminated from consideration. A step 148 uses spectrum analysis as well as local contrast to distinguish the seeds, which have adjacent mirror structures, from other bright parts, such as those caused by calcification and/or air bubbles. Calcified structures have rough surfaces that do not produce an adjacent mirror structure. This step finds candidate position of the seeds and uses a window size of 16 pixels instead of six, as in step 144. FIG. 13C illustrates an example showing a TRUS image 280 in which the bright parts caused by non-seed structures have been eliminated, identifying the remaining bright elements (within the white circles) as seeds.

In step 148, a signal of 16-pixel length is synthesized by:

s(n)=−(sin(0.15 πn)+sin(0.3 πn)) (1)

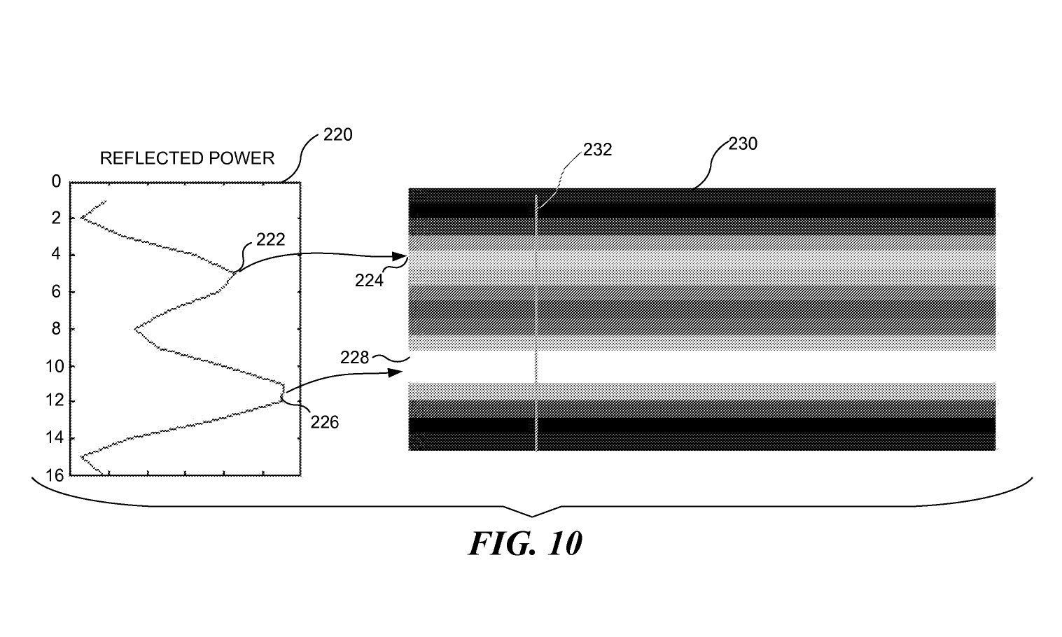

and an image pattern is created by this signal. Every vertical line in the pattern is a linear mapping from s(n) to a range from 0 to 255, and the pattern is 30 pixels wide. FIG. 10 illustrates how this signal of the mirror structure is modeled. A synthesized image 220 has a pattern of peaks 222 and 226 similar to that of the seed in TRUS images and is used as the model of a corresponding mirror structure 230 along a line 232, as shown in FIG. 10. Peak 222 corresponds to a bright line structure 224, while peak 226 corresponds to a bright line structure 228.

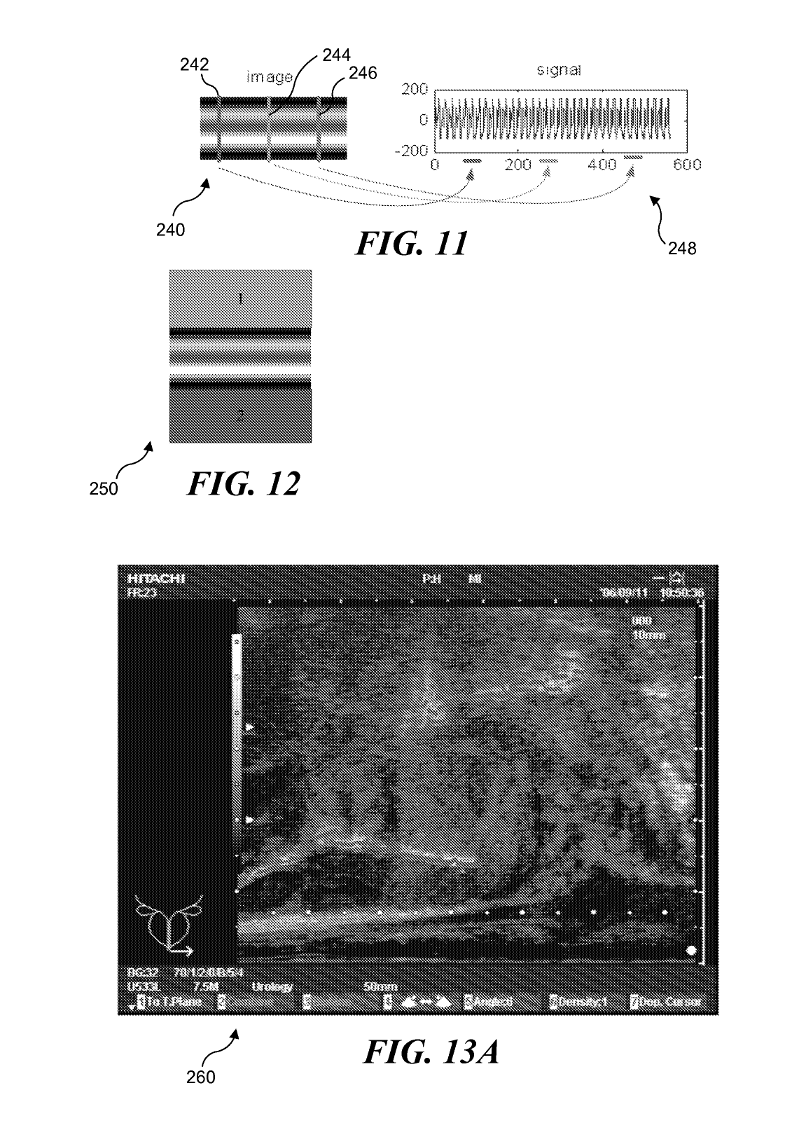

Because the image signal is too short for spectrum analysis, it is necessary to extend a 2-D image pattern 240 having lines 242, 244, and 246 to a 1-D signal 248, as shown in FIG. 11. Some tissue may have a power spectrum similar to that of a seed. It is hard to distinguish between such tissue and seeds by spectrum analysis. Accordingly, the average brightness of the seed area is determined and is compared with the average brightness of Region 1 and Region 2, which are respectively the two 2-D windows above and below a seed structure, as shown in an image 250 in FIG. 12.

Only a structure with high local contrast is considered as a seed. While other techniques might be used for carrying out spectrum analysis of the 1-D signal produced by extending the 2-D image patterns, experiments have shown that the multiples signal classification (MUSIC) algorithm provides good results for detecting seeds in TRUS images, as indicated in a step 148 of FIG. 5.

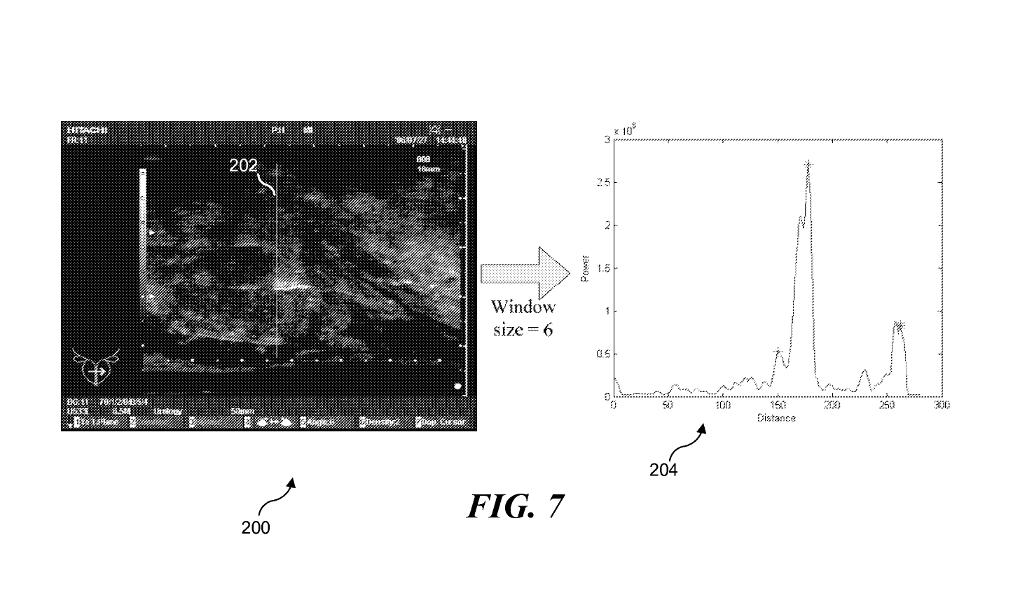

In these preceding steps, each vertical line in the TRUS image is treated as a scan line of the ultrasound beam. The smooth seed surface should produce a specular reflection of the ultrasound beam, so the power at the seed is relatively great compared to the background power. In step 144 of FIG. 5, a window is slid along a vertical line, and the power in every window position is calculated. To find the precise location of a seed and to reduce the effect of noise, a six-pixel wide window is used in this step. FIG. 7 illustrates an exemplary TRUS image 200, and peaks 204 detected along a six-pixel wide window that lies along a vertical line 202 in the image. In selecting candidate seed positions, the procedure identifies local peaks, since a seed must be brighter than the tissue near it. Only the peaks that are higher than a maximum background level in a line can be a candidate seed position.

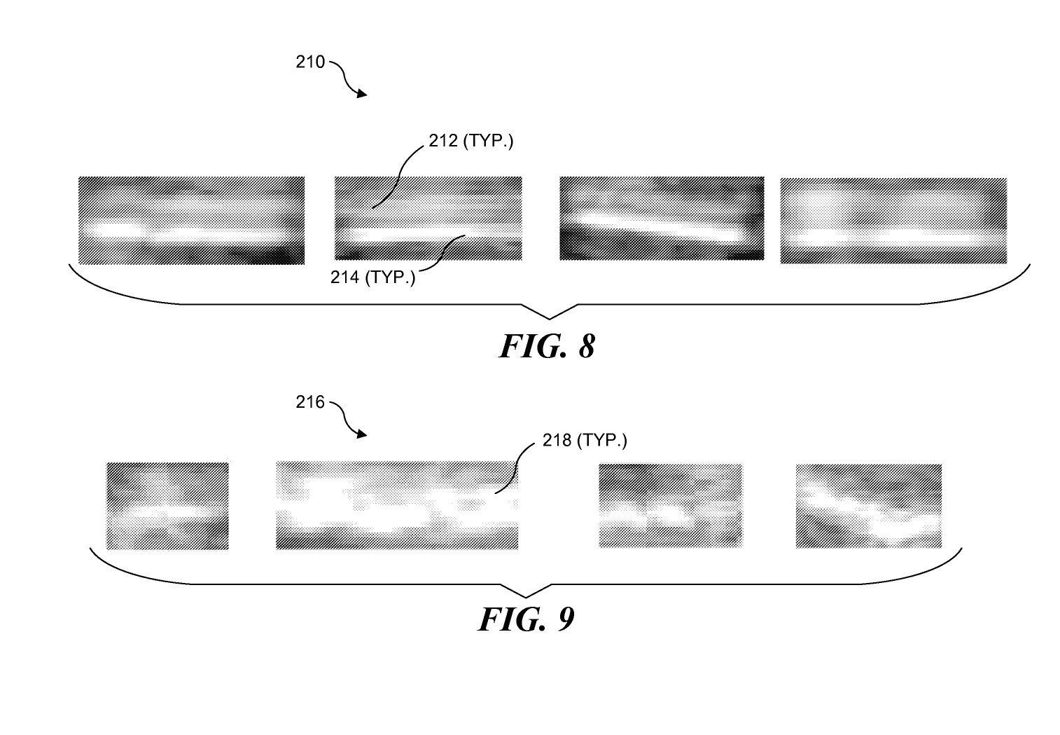

In clinical TRUS images, the non-seed structures (calcification and/or air bubbles) in the prostate may appear as a bright line structure similar to that of seeds. A non-seed structure can be distinguished from a seed not by its own appearance, but because a seed has a mirror structure above it that does not exist in a calcification and/or air bubble trapped along a needle track. In TRUS images, a seed appears to have a mirror above it that is caused by the specular reflection between the two smooth surfaces of the seed. A non-seed structure does not exhibit this characteristic, because its surface is rough, and no specular reflection happens on the rough surface. Accordingly, if two peaks on a line are close to each other and the one nearer to the transducer has a higher value, this peak is selected as identifying a seed position, because the other peak is caused by the mirror effect of the seed. FIG. 8 illustrates exemplary TRUS images 210 of several seeds 214 and their mirror reflections 212. In contrast, FIG. 9 illustrates several exemplary TRUS images 216 showing calcifications and/or air bubbles 218, which clearly do not exhibit the mirror structure.

In a step 150 of FIG. 5, a constraint is applied between adjacent frames (i.e., different frames) so that only the structures that appear at the same location in several adjacent frames are considered as actual seeds, but will only be counted once. The resulting seed location(s) in the image are returned in a step 152 and comprise a subset of the total number of seeds implanted. Experimental results have shown that 20% or more of the total number of seeds that have been implanted can thus be automatically detected in the TRUS longitudinal images. It should be noted that although the steps of FIG. 5 are used for automatically detecting the subset or minority of the implanted seeds, the present approach can also be carried out after an experienced medical practitioner has manually identified the locations of seeds comprising a subset of the total implanted seeds by visual inspection of the TRUS rotational/longitudinal images.

### IOA Registration of Model Set and Data Set

An exemplary approach used in connection with the present novel technology uses IOA to achieve registration between the model set (i.e., the locations of all of the implanted seeds as determined from the fluoroscopic images), and the data set (the locations of the subset of implanted seeds determined by analysis of the TRUS images). IOA is similar to a prior art approach called “iterative closest point” (ICP) in the sense that transformation parameters and a correspondence matrix are used to refine each other in each iteration. However, recognizing the weakness of the nearest-neighbor approach, correspondence establishment is formulated as an optimal assignment problem that is solved with the Hungarian method. Thus, for a given set of seed pose parameters, a one-to-one correspondence with a minimum associated cost is established. Then, IOA searches iteratively through the parameter space to find the pose parameters that provide the best alignment between the data set and model set.

The following describes the IOA algorithm implemented using the Hungarian method. An assignment A is a one-to-one mapping from K to L. Since |K|≦|L|, for each kεK, there exists a match lεL, where I=a(k). Let q(k, l) be the cost of associating kεK with IεL, then the cost of an assignment A is given as:

$$q ( A ) = {\sum }_{k ∈ K} q ( k , a ( k ) ) . \qquad ( 2 )$$

The optimal assignment problem involves minimizing q(A) over all assignments under the assumption that |K|=|L|. If |K|≦|L|, |L|−|K| dummy elements should be added to the data set such that the cost of associating the newly added elements with every lεL is zero. Let Q be an |L|×|L| matrix with elements q(k, l) after augmenting K with dummy elements as described above. In this setting, the optimal assignment is reduced to minimizing the sum of |L| elements chosen from Q such that no two chosen elements are on the same column or row. A brute-force algorithm can solve this problem by computing the cost of all |L|! possible assignments and finding the minimum. However, this approach has an exorbitantly large computational cost.

The algorithm known as the Hungarian method, which was proposed by Kuhn based on the early works of two Hungarian mathematicians, Egervary and Konig, and later modified by Munkres, can solve the optimal assignment problem with O(|K|²|L|) run-time complexity. Since the Hungarian method is well known in the art, it is unnecessary to describe it in detail herein. Knuth's implementation was used in an exemplary embodiment.

### Iterative Pose Estimation

Using the terminology introduced above, the registration problem can be rephrased as follows. Find a set of transformation parameters, ξ, such that after applying the corresponding transformation on the model set, the total cost of the resulting optimal assignment between the data and model sets is minimal.

Typically, a transformation is applied on the data set. However, to perform dosimetry, it is necessary to superimpose the seeds that have been reconstructed from fluoroscopic images on the axial TRUS images. Therefore, in the present case, the model set is transformed while the data set is kept stationary. Let (x, y, z) be the data reference frame and (x′, y′, z′) be the model reference frame. The optimum pose parameters can be found by minimizing the following criterion function:

$${ξ}^{*} = {arg min}_{ξ} ( {\sum }_{k ∈ K} \vert F ( ξ ) [ \begin{matrix}{x}^{′} ( a ( k ) ) \\ {y}^{′} ( a ( k ) ) \\ {z}^{′} ( a ( k ) )\end{matrix} ] - [ \begin{matrix}x ( k ) \\ y ( k ) \\ z ( k )\end{matrix} ] \vert ) , \qquad ( 3 )$$

where F (ξ) is given as the transformation matrix. In the case of rigid-body registration, the pose parameter set consists of six parameters, xₒ, yₒ, zₒ for translation and κ, φ, ω for rotation. Then, the transformation matrix is given as:

$$F ( ξ ) = \begin{matrix}[ \begin{matrix}cos κ & - sin κ & 0 \\ sin κ & cos κ & 0 \\ 0 & 0 & 1\end{matrix} ] [ \begin{matrix}cos φ & 0 & sin φ \\ 0 & 1 & 0 \\ - sin φ & 0 & cos φ\end{matrix} ] \times \\ [ \begin{matrix}1 & 0 & 0 \\ 0 & cos ω & - sin ω \\ 0 & sin ω & cos ω\end{matrix} ] [ \begin{matrix}1 & 0 & 0 & - {x}_{0} \\ 1 & 0 & 0 & - {y}_{0} \\ 1 & 0 & 0 & - {z}_{0}\end{matrix} ] .\end{matrix} \qquad ( 4 )$$

IOA attempts to find the set of optimum pose parameters ξ* in Eq. (3) based on the flowchart shown in FIG. 6. The optimization loop finds the set of pose parameters associated with the minimum cost around a given set of initial pose parameters. Powell's method is used to perform the optimization. In the first iteration, the rotational parameters are set to zero, and the model set is translated such that its centroid overlaps with that of the data set. However, if the initial set of pose parameters is far away from the global solution, the cost function may not be convex, and Powell's method, like most other optimization methods, can converge to a local solution in the parameter space. Therefore, to increase the likelihood of finding the global minimum, IOA repeats the optimization loop hundreds of times. At each iteration, a different set of initial pose parameters is sampled from a specified interval.

Exemplary logical steps 160 of the IOA procedure are illustrated in FIG. 6. In a step 162, the model set is input, while in a step 164, the data set is input. As explained above, the model set comprises the locations of all of the implanted seeds, derived from the fluoroscopic images, while the data set comprises the subset or minority of the seed positions that were identified in the TRUS longitudinal images. The model set is input to a step 166, which performs a 3-D rigid body transformation of the input information using an estimated seed pose parameter set. The data set is input to a step 168, which also receives the 3-D rigid body transformation from step 166. Step 168 computes an optimal assignment between the data set and the model set (transformed) using the Hungarian method. A decision step 170 then determines if the solution has converged, and if not, proceeds to a step 172, which adjusts the pose parameter set previously used to attempt to reach a convergence. The adjusted pose parameter set is then input to step 166 to redo the 3-D rigid body transformation, and the algorithm loops back to step 168 with a revised 3-D rigid body transformation of the model set. This optimization loop repeats until the solution converges in decision step 170 and then proceeds with a step 174, which records the solution ξᵢ′ around the pose parameter set ξᵢ, and its associated cost Qᵢ. A decision step 176 then determines if the index i is greater than the number of iterations, T. If not, a step 178 increments the index i by one before proceeding with a step 180, which samples a new pose parameter set, ξᵢ. (Given an initial set of pose parameters, the Hungarian algorithm may be called several times inside the optimization loop. T is the maximum number of iterations that the optimization loop can be called. i.e., T specifies how many times an initial set of pose parameters should be generated in order to find the solution around it.) From step 180, the logic reenters the optimization loop at step 166. Once the determination in decision step 176 is affirmative, the logic continues with a step 182, which exits the IOA procedure with a then current optimal seed pose parameter set ξₖ′, such that Qₖ is equal to the minimum of (Qᵢ . . . Q_{T}).

### Clinical Evaluation of Technique

The present approach for determining dosage parameters while a patient is undergoing prostate brachytherapy to enable additional seeds to be implanted has been implemented during tests conducted on 25 consecutive patients. These 25 unselected patients with American Joint Commission on Cancer clinical state T1c-T2 prostate cancer were treated with Pd-103 seed implantation, and the dosimetry was evaluated using the real-time postimplant fusion of TRUS and fluoroscopically derived isodoses, to enable intraoperative corrections to maximize dosimetric parameters that were achieved. The isodose data were determined based on using the procedure described above.

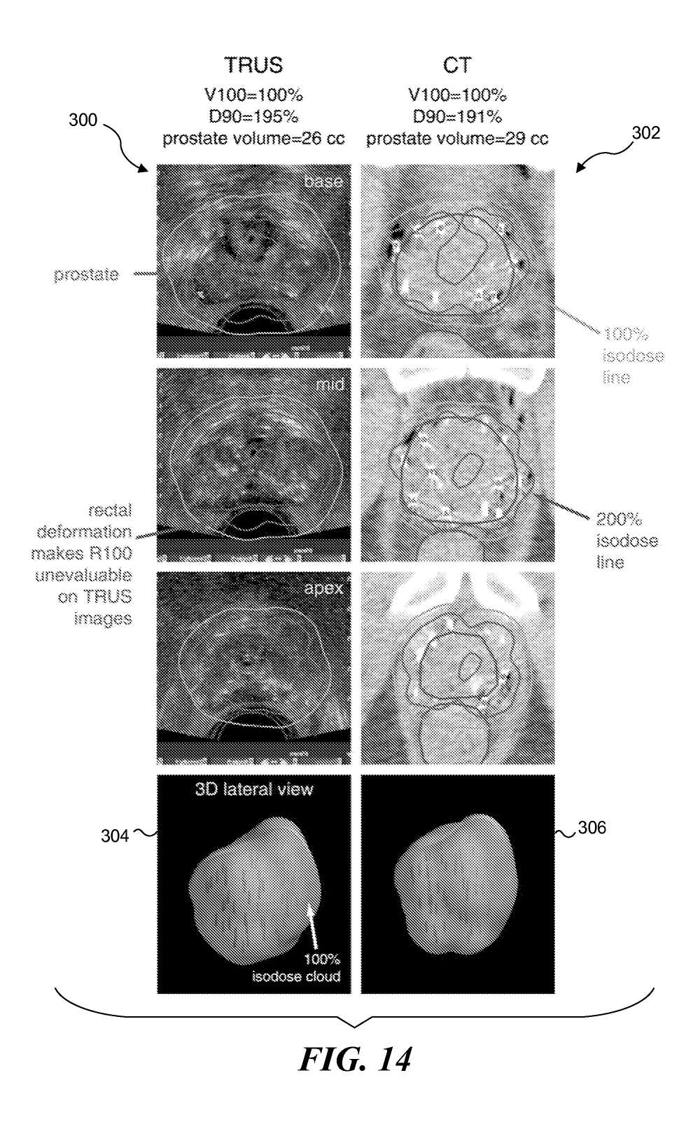

FIG. 14 illustrates a comparison of the intraoperative dosimetry that was determined using the present approach (shown in images 300 on the left), and Day 0 computed tomography (CT) based dosimetry (shown in images 302 on the right). The prostate contours are identified in the cross-sectional images, along with V200, V100 isodose contours. Also included are a TRUS 3-D lateral view image 304 showing 100% isodose cloud, and a corresponding CT view 306. The prostate volume in this example was determined to be 26 ml in the TRUS images, and 29 ml in the CT images. The V100 value was found to be 100% in both the intraoperative and CT-based postoperative dosimetry studies, whereas D90 values were 195% and 191%, respectively.

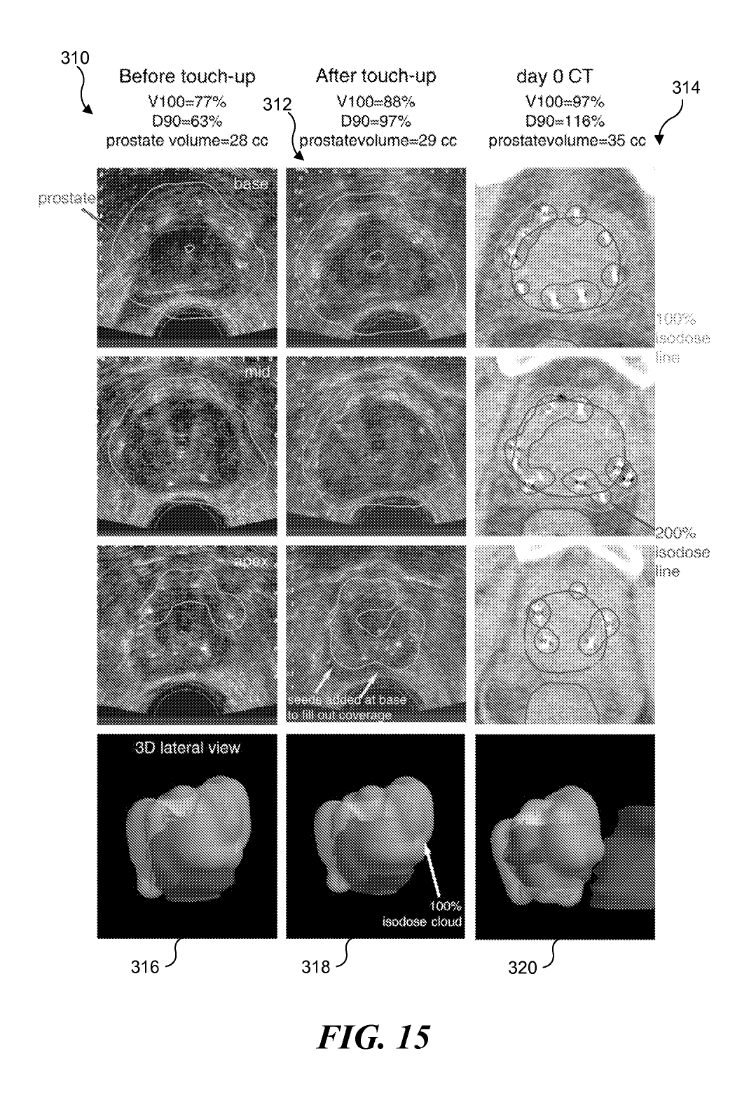

FIG. 15 illustrates initial and corrected intraoperative dosimetry compared with Day 0 CT-based dosimetry. Again, the prostate contour is identified in these cross-sectional images, along with the V200 and V100 isodose contours. The left column in this Figure includes TRUS images 310 that illustrate the intraoperative isodose contours determined before touch-up; the middle column includes TRUS images 312 that indicate the intraoperative isodose contours after additional seeds were implanted; and, the right column includes Day 0 CT images 314 that were produced after implant of the additional seeds. The transparent cloud in the three-dimensional visualization encloses the volume that receives the prescribed dose. Also included are 3-D lateral views 316 and 318, for the TRUS images before and after implant of the additional seeds, and a corresponding CT view 320 after the additional seeds were implanted. The prostate volume was determined as 28 ml, 29 ml, and 35 ml in the initial intraoperative, corrected intraoperative, and CT-based postoperative dosimetry studies, respectively. The comparison of initial and corrected intraoperative results shows an improvement in V100 from 77% to 88%, and an increase in D90 from 63% to 97%. The V100 and D90 values were computed as 97% and 116% in the postoperative CT-based dosimetry.

In nine of the patients, no seeds were added after the intraoperative dose evaluation. Their average intraoperative V100s and D90s were 99±1.5% and 176±23%. In 16 patients, an average of 4±1.8 additional seeds was added, based on the initial intraoperative evaluation. The initial intraoperative V100 and D90s were 86±8% and 94±18%, respectively. The corrected intraoperative V100 and D90s were 93±4% and 109±12%, respectively. The average improvement in the V100 and D90 parameters was 7.0% (p=0.005) and 8% (p=0.011), respectively. The V200s and V300s were minimally affected.

Imaging System for Prostate Brachytherapy with Intraoperative Dosimetry

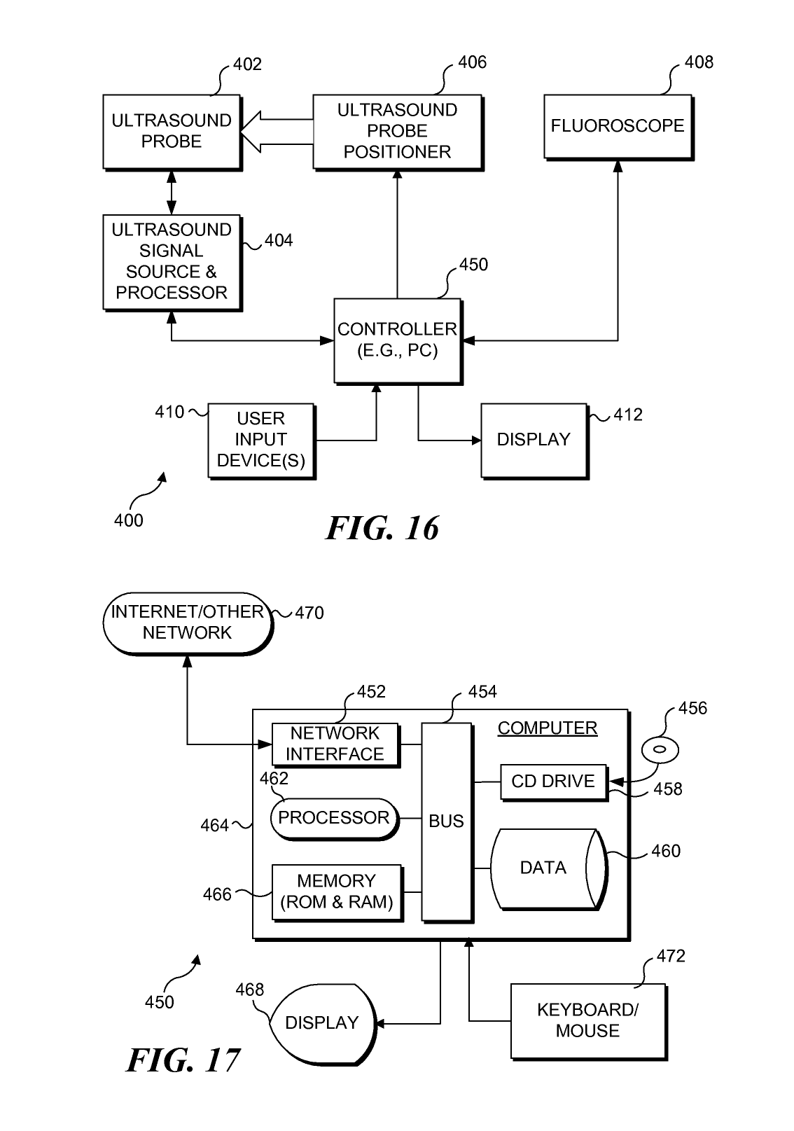

A combined ultrasound and fluoroscopic imaging system 400 for providing prostate brachytherapy with intraoperative dosimetry, as described above, is illustrated in FIG. 16. This system includes an ultrasound probe, which may be a conventional TRUS probe 402 (or alternatively, a 3-D ultrasound probe or other movable probe that is capable of providing both longitudinal and axial imaging of the prostate). The ultrasound probe is driven with an input signal from an ultrasound signal source and processor 404 and is moved longitudinally, and/or rotationally by an ultrasound probe positioner 406, which includes one or more stepping motors (not shown) that serve as prime movers. Not shown are position encoders for providing data indicative of the position of the probe when imaging.

Also included in system 400 is a conventional fluoroscope 408 that uses X-rays to image the prostate region to provide images showing the disposition of all implanted seeds. Output signals from ultrasound signal source and processor 404 and from fluoroscope 408 are input to a controller 450, which may be a computing device—either hardwired or programmed, such as a personal computer (PC). This controller also controls the fluoroscope and ultrasound system, as well as ultrasound probe positioner 406, so that ultrasound probe 402 collects the desired ultrasound images in which the prostate and disposition of each of a subset of the implanted seeds are indicated, and the fluoroscope collects the images in which the disposition of all of the implanted seeds is indicated. Controller 450 processes both of these types of images to produce the model set and data set discussed above, and then determines the registration between the two types of images so that dosage parameters can be determined intraoperatively. The medical practitioner can provide text or make selections with one or more user input devices 410. The images of the prostate region, as well as isodose contours relative to axial views of the prostate can be displayed on a display 412, enabling the medical practitioner to intraoperatively assess whether additional seeds should be implanted and where they should be implanted to achieve an optimal treatment of the prostate, as explained above.

### Exemplary Computing Device for Use in Practicing the Present Novel Method

FIG. 17 schematically illustrates an exemplary computing system (or controller) 450 suitable for implementing the present novel technique. While other forms of logic devices can be employed, computing system 450 can include a computer 464 that may be a generally conventional personal computer (PC) such as a laptop, desktop computer, or other form of computing device. Computer 464 is coupled to a display 468, which is used for displaying text and graphics to the user. Included within computer 464 is a processor 462. A memory 466 (with both read only memory (ROM) and random access memory (RAM)), a non-volatile storage 460 (such as a hard drive or other non-volatile data storage device) for storage of data, digital signals, and software programs, a network interface 452, and an optical drive 458 are coupled to processor 462 through a bus 454. Optical drive 458 can read a compact disk (CD) 456 (or other optical storage media, such as a digital video disk (DVD)) on which machine instructions are stored for implementing the present novel technique, as well as other software modules and programs that may be run by computer 464. The machine instructions are loaded into memory 466 before being executed by processor 462 to carry out the steps for implementing the present technique.

The user employs computer 464 to process the images produced by the fluoroscopic system and the ultrasound probe. Processor 462 executes the machine instructions stored in memory 466. These machine instructions cause the processor to determine the model set and the data set from the fluoroscopic images and TRUS images, respectively, and then based upon the registration of these sets, determines dosage parameters and/or isodose contours that can be viewed on display 468. The results can be stored on storage 460, or on a separate storage—not shown in FIG. 17, which can be accessed by a connection to the Internet/other network 470 through network interface 452. The user can interact with and provide input to computer 464 using a keyboard/mouse 472.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for performing a prostate brachytherapy procedure on a patient, in which radioactive seeds are implanted at a treatment site in the patient, and for intraoperatively determining a dosage parameter for implanted seeds during the procedure, comprising the steps of:
(a) implanting a plurality of radioactive seeds at the treatment site;
(b) acquiring X-ray data that identify locations of a majority of the implanted seeds;
(c) acquiring ultrasound data that identify a location of the prostate, and locations of a minority of the implanted seeds relative to the prostate, wherein the minority of implanted seeds and their locations relative to the prostate are identified without solely relying on cross-sectional images of the seeds;
(d) registering the X-ray data with the ultrasound data to determine locations of the majority of the implanted seeds relative to the prostate; and
(e) determining a dosage parameter for the implanted seeds based on the locations of the majority of the seeds relative to the prostate.

2. The method of claim 1, wherein the ultrasound data include longitudinal ultrasound images extending along an axis that is generally aligned with a longitudinal axis of each implanted seed, and wherein the step of identifying the minority of seeds comprises the steps of:
(a) identifying bright structures in the longitudinal ultrasound images having a reflected ultrasound power level that is substantially greater than a background power level;
(b) detecting whether the bright structures identified in the longitudinal ultrasound images correspond to line structures; and
(c) detecting each of the line structures that includes an adjacent mirror structure reflection that is indicative of the line structure being an implanted seed.

3. The method of claim 1, wherein the step of acquiring the ultrasound data comprises the step of collecting both axial ultrasound images and longitudinal ultrasound images, wherein the longitudinal ultrasound images are generally aligned with a longitudinal axis of the implanted seeds and the axial ultrasound images are generally transverse to the longitudinal axis of the implanted seeds.

4. The method of claim 1, wherein the step of acquiring the ultrasound data comprises the step of either:
(a) rectally inserting an ultrasound probe to enable transrectal ultrasound (TRUS) images of the treatment site to be collected in orthogonally different orientations; or
(b) inserting the ultrasound probe in a urethral passage of a patient, to enable transurethral ultrasound images of the treatment site to be collected in orthogonally different orientations.

5. The method of claim 1, wherein the step of acquiring the X-ray data comprises the step of collecting a plurality of fluoroscopic images of the treatment site at different angular orientations, after the seeds have been implanted, so that the majority of the implanted seeds can be identified in the plurality of the fluoroscopic images.

6. The method of claim 1, wherein the step of determining a dosage parameter for the implanted seeds comprises the step of determining isodose contours for the implanted seeds relative to the prostate, further comprising the steps of:
(a) displaying the isodose contours of the implanted seeds in relation to the prostate; and
(b) enabling a medical practitioner viewing the isodose contours to intraoperatively determine during the procedure whether additional seeds should be implanted and if so, where the additional seeds should be implanted to achieve a desired radiation dosage effect on the prostate.

7. The method of claim 1, wherein the step of registering the X-ray data with the ultrasound data comprises the step of iteratively performing an optimization loop that transforms the X-ray data and correlates transformed X-ray data with the ultrasound data.

8. The method of claim 7, wherein the step of iteratively performing the optimization loop comprises the steps of:
(a) performing a rigid body transformation of the X-ray data based on a current pose parameter set, producing the transformed X-ray data;
(b) computing an optimal assignment of the transformed X-ray data to the ultrasound data; and
(c) until a solution to the optimal assignment converges, repetitively adjusting the current pose parameter set and carrying out the steps of performing the rigid body transformation and computing the optimal assignment.

9. The method of claim 8, wherein the step of registering the X-ray data with the ultrasound data further comprises the steps of:
(a) recording the solution that has converged and associated costs of said solution;
(b) until the optimization loop has been entered a number of times that is greater than a predefined maximum number of iterations, carrying out the following steps:
(i) sampling a new pose parameter set for use as the current pose parameter set;
(ii) reentering the optimization loop with the new pose parameter set that now comprises the current pose parameter set;
(iii) iteratively repeating the steps comprising the optimization loop until the solution again converges; and
(iv) recording a current solution that has converged and a current associated cost of said solution; and
(c) exiting with the current solution and the current associated cost after the optimization loop has been entered more times than the predefined maximum number of iterations.

10. A system for performing a prostate brachytherapy procedure on a patient, in which radioactive seeds are implanted at a treatment site in the patient, and for intraoperatively determining a dosage parameter for implanted seeds during the procedure, comprising:
(a) an ultrasound system that includes a probe configured to produce ultrasound data corresponding to volumetric images of the treatment site, or to both axial and longitudinal ultrasound images of the treatment site, the longitudinal images being generally aligned with a longitudinal axis of seeds that have been implanted and the axial images being generally transverse to the longitudinal axis of the seeds that have been implanted, the ultrasound data being capable of indicating locations of a minority of the seeds that have been implanted relative to the prostate of the patient;
(b) a fluoroscope that is configured for producing X-ray data for the treatment site, for indicating a majority of the seeds that have been implanted; and
(c) a computing device for processing the ultrasound data and the X-ray data, said computing device carrying out a plurality of functions, including:
(i) processing the X-ray data to identify the majority of the implanted seeds;
(ii) processing the ultrasound data to identify a location of the prostate, and to identify locations of each of the minority of the implanted seeds relative to the prostate;
(iii) registering the majority of the implanted seeds acquired from the X-ray data with the locations of the minority of the implanted seeds acquired from the ultrasound data to determine locations of the majority of the implanted seeds relative to the prostate; and
(iv) determining a dosage parameter for the implanted seeds based on the locations of the majority of the seeds relative to the prostate.

11. The system of claim 10, wherein the computing device identifies the locations of the minority of seeds that are implanted by:
(a) identifying bright structures in the longitudinal ultrasound images having a reflected ultrasound power level that is substantially greater than a background power level;
(b) detecting whether the bright structures identified in the ultrasound images correspond to line structures; and
(c) detecting each of the line structures having an adjacent mirror structure reflection, wherein each line structure having an adjacent mirror structure reflection is identified as being an implanted seed.

12. The system of claim 10, wherein the ultrasound probe is configured either to be rectally inserted into a patient's body, for collecting transrectal ultrasound (TRUS) data of the treatment site, or to be inserted along a urethral passage of the patient's body, for collecting transurethral ultrasound data of the treatment site.

13. The system of claim 12, further comprising an ultrasound probe positioner that moves the ultrasound probe along a longitudinal axis of the ultrasound probe and rotates the ultrasound probe about said longitudinal axis.

14. The system of claim 10, wherein the fluoroscope is positionable at a plurality of different positions around a craniocaudal axis of a patient's body, to collect the X-ray data, which comprises a plurality of fluoroscopic images of the treatment site.

15. The system of claim 10, wherein the dosage parameter determined by the computing device comprises isodose contours for the implanted seeds relative to the prostate, further comprising a display on which the computing device displays the isodose contours to enable a medical practitioner viewing the isodose contours to intraoperatively determine whether additional seeds should be implanted and if so, where the additional seeds should be implanted to achieve a desired radiation dosage effect on the prostate.

16. The system of claim 10, wherein the computing device registers the majority of the implanted seeds acquired from the X-ray data with the locations of the minority of the implanted seeds acquired from the ultrasound data by iteratively performing an optimization loop that transforms the X-ray data and correlates transformed X-ray data with the ultrasound data.

17. The system of claim 16, wherein the computing device iteratively executes the optimization loop by:
(a) performing a rigid body transformation of the X-ray data based on a current pose parameter set, producing the transformed X-ray data;
(b) computing an optimal assignment of the transformed X-ray data to the ultrasound data; and
(c) until a solution to the optimal assignment converges, repetitively adjusting the current pose parameter set and carrying out the steps of performing the rigid body transformation and computing the optimal assignment.

18. The system of claim 17, wherein the computing device further registers the majority of the implanted seeds acquired from the X-ray data with the locations of the minority of the implanted seeds acquired from the ultrasound data by:
(a) recording the solution that has converged and associated costs of said solution;
(b) until the optimization loop has been entered a number of times that is greater than a predefined maximum number of iterations, carrying out the following steps:
(i) sampling a new pose parameter set for use as the current pose parameter set;
(ii) reentering the optimization loop with the new pose parameter set that now comprises the current pose parameter set;
(iii) iteratively repeating the steps comprising the optimization loop until the solution again converges; and
(iv) recording a current solution that has converged and a current associated cost of said solution; and
(c) exiting with the current solution and the current associated cost after the optimization loop has been entered more times than the predefined maximum number of iterations.

19. A method for identifying radioactive seeds implanted at a treatment site, by automated processing of ultrasound data collected for the treatment site, comprising the steps of:
(a) identifying bright structures in the ultrasound data having a reflected ultrasound power level that is substantially greater than a background power level;
(b) detecting whether the bright structures identified in the ultrasound data correspond to line structures; and
(c) detecting each of the line structures that includes an adjacent mirror structure reflection, wherein the mirror structure reflection is characteristic of a specular reflection from a seed and thereby indicates that the line structure is likely an implanted seed, to identify a location of at least a portion of the seeds implanted at the treatment site.

20. The method of claim 19, wherein the mirror structure reflection is of a lower power level than an adjacent reflection from an implanted seed, further comprising the step of automatically excluding reflections from non-seed structures, including calcifications and air bubbles, as not being from an implanted seed, because the non-seed structures are rough and do not produce a specular reflection corresponding to the mirror structure reflection.

21. The method of claim 19, further comprising the step of determining whether a line structure identified as likely being a seed appears at a same location in a plurality of adjacent longitudinal ultrasound image frames, and if so, identifying the line structure as an implanted seed.

22. The method of claim 19, wherein the step of detecting whether the bright structures identified in the ultrasound data correspond to line structures comprises the step of applying either an 8-connected neighborhood transform or a Hough transform to image frames of the ultrasound data, either of said transforms being capable of detecting line structures that may be implanted seeds.

23. A system for identifying radioactive seeds implanted at a treatment site, by automated processing of ultrasound data collected for the treatment site, comprising:
(a) a processor that is adapted to receive ultrasound data collected for a treatment site at which radioactive seeds have been implanted, the ultrasound data being usable to produce longitudinal ultrasound images that are generally aligned with a longitudinal axis of implanted seeds; and
(b) a memory in which machine instructions are stored for implementing a plurality of functions when executed by the processor, including:
(i) identifying bright structures in the ultrasound data having a reflected ultrasound power level that is substantially greater than a background power level;
(ii) detecting whether the bright structures identified in the ultrasound data correspond to line structures; and
(iii) determining whether any of the line structures detected includes an adjacent mirror structure reflection, wherein the adjacent mirror structure reflection is characteristic of specular reflection from a seed and thereby indicates that the line structure is likely an implanted seed, to identify locations of at least a portion of the seeds implanted at the treatment site.

24. The system of claim 23, wherein the mirror structure reflection is of a lower power level than an adjacent reflection from an implanted seed, and wherein the machine instructions further cause the processor to automatically exclude reflections from non-seed structures as not being from an implanted seed, because the non-seed structures are rough and do not produce a specular reflection corresponding to the mirror structure reflection.

25. The system of claim 23, wherein the machine instructions further cause the processor to determine whether a line structure identified as likely being a seed appears at a same location in a plurality of adjacent longitudinal ultrasound image frames, and if so, identifying the line structure as an implanted seed.

26. The system of claim 23, wherein the machine instructions further cause the processor to detect whether the bright structures identified in the ultrasound data correspond to line structures by applying either an 8-connected neighborhood transform or a Hough transform to image frames of the ultrasound data, either of said transforms being capable of detecting line structures that may be implanted seeds.

27. A method for registering a subset of radioactive seeds implanted at a treatment site that have been identified in ultrasound data with a set of the radioactive seeds that have been identified in X-ray data, so that a spatial disposition of the set of seeds can be determined relative to a tissue portion of a patient's body, comprising the steps of:
(a) providing the X-ray data and the ultrasound data as input to an optimization loop that transforms the X-ray data and correlates resulting transformed X-ray data with the ultrasound data;
(b) iteratively entering the optimization loop until a predefined condition is met; and
(c) once the predefined condition has been met, exiting the step of iteratively entering the optimization loop with a final pose parameter set that registers the X-ray data with the ultrasound data, such that when a cost associated with the final pose parameter set is a minimum, a disposition of each implanted seed relative to the tissue portion of the patient's body is defined by the final pose parameter set.

28. The method of claim 27, wherein a step of implementing the optimization loop comprises the steps of:
(a) performing a rigid body transformation of the X-ray data based on a current pose parameter set, producing the transformed X-ray data; and
(b) computing an optimal assignment of the transformed X-ray data to the ultrasound data.

29. The method of claim 28, further comprising the steps of:
(a) iteratively repeating the optimization loop until a solution to the optimal assignment converges; and
(b) before each repetition of the optimization loop, repetitively adjusting a current pose parameter set used for the step of performing the rigid body transformation.

30. The method of claim 28, wherein the step of computing the optimal assignment of the transformed X-ray data to the ultrasound data comprises the step of applying the Hungarian method to determine the optimal assignment.

31. The method of claim 28, if the solution within the optimization loop has not yet converged, further comprising the step of adjusting a current pose parameter set before repeating the step of performing the rigid body transformation of the X-ray data.

32. The method of claim 27, wherein the predefined condition corresponds to a number of entries into the optimization loop exceeding a number of times that the optimization loop has been iterated.

33. A system for registering a subset of radioactive seeds implanted at a treatment site that have been identified in ultrasound data with a set of the radioactive seeds that have been identified in X-ray data, so that a spatial disposition of the set of seeds can be determined relative to a tissue portion of a patient's body, comprising:
(a) a processor that is adapted to receive both the ultrasound data and the X-ray data collected for the treatment site; and
(b) a memory in which machine instructions are stored, for implementing a plurality of functions when executed by the processor, including:
(i) executing an optimization loop that transforms the X-ray data and correlates resulting transformed X-ray data with the ultrasound data;
(ii) iteratively entering the optimization loop until a predefined condition is met; and
(iii) once the predefined condition has been met, providing a final pose parameter set that registers the X-ray data with the ultrasound data, such that when a cost associated with the final pose parameter set is a minimum, disposition of each implanted seed relative to the tissue portion of the patient's body is defined by the final pose parameter set.

34. The system of claim 33, wherein the machine instructions further cause the processor to:
(a) iteratively repeat execution of the optimization loop until a solution to the optimal assignment converges; and
(b) before each repetition of the optimization loop, repetitively adjust a current pose parameter set used to transform the X-Ray data.

35. The system of claim 33, wherein the machine instructions cause the processor to compute the optimal assignment of the transformed X-ray data to the ultrasound data by applying the Hungarian method to determine the optimal assignment.

36. The system of claim 33, wherein if the solution within the optimization loop has not yet converged, the machine instructions cause the processor to adjust a current pose parameter set before again performing the rigid body transformation of the X-ray data.

37. The system of claim 32, wherein the predefined condition corresponds to a number of entries into the optimization loop exceeding a number of times that the optimization loop has been iterated.
